(19) United States
(12) Patent Application Publication
(10) Pub. No.: US 2004/0098129 A1
(43) Pub. Date: May 20, 2004

(54) Spinal implant insertion adjustment instrument and implants for use therewith

(76) Inventor: Jo-Wen Lin, Tinton Falls, NJ (US)

Correspondence Address: William Squire, Esq., c/o Carella, Byme, Bain, Gilfillan,, Cecchi, Stewart & Olstein, 5 Becker Farm Road, Roseland, NJ, 07068, US

(21) Appl. No.: 10/700,200

(22) Filed: Nov. 3, 2003

Related U.S. Application Data
(60) Provisional application No. 60/425,941, filed on Nov. 13, 2002.

(51) Int. Cl. A61F 2/44

(52) U.S. Cl. 623/17.11; 606/61; 623/17.16

(57) ABSTRACT

A spinal implant of cortical bone for insertion in the anterior to antereolateral approaches has a smooth surfaced cylindrical bore along a plane between inferior and superior vertebral engagement surfaces which may be roughened and which may be inclined to match the lordosis of the vertebrae. The bore may extend from the anterior surface through the implant into a central chamber or may be blind. The instrument has a straight shaft which is bent at a proximal end opposite a handle at the distal end. The bend is formed into an implant engaging member which has a circular cylindrical smooth surface and closely mates with and is complementary to the implant bore. The instrument is used to rotate or otherwise finely manipulate the implant in the plane of the disc space to a desired orientation from the initial improper insertion orientation. Impact forces on the handle torques the instrument and implant in the desired direction with minimum stress concentration on the implant in the bore to minimize damage to the bone that might otherwise occur in the presence of such stress concentration.

This application claims the benefit of provisional application Ser. No. 60/425,941 filed Nov. 13, 2002 which is incorporated by reference herein in its entirety.

This invention relates to spinal intervertebral fusion implants and insertion instruments for insertion of the implant into the intervertebral disc space, and more particularly, to anterior approach implants and instruments.

### CROSS REFERENCE TO RELATED APPLICATIONS AND PATENTS

Of interest are commonly owned copending provisional applications Ser. No. 60/340,734 filed Oct. 30, 2001 and Ser. No. 60/372,972 filed Apr. 16, 2002, both in the name of John Winterbottom et al., both applications corresponding to US utility application Ser. No. 10/282552 filed Oct. 29, 2002 (attorney docket 525400-284) and PCT application Ser. No. PCT/U.S. 02134466 filed Oct. 28, 2002 (attorney docket 525400-283), Ser. No. 10/086041 entitled Spinal Intervertebral Implant Insertion Tool filed Oct. 25, 2001 in the name of Erik Martz et al. and Ser. No. 10/046866 entitled Implant Insertion Tool filed Jan. 15, 2002 in the name of John M. Winterbottom et al., and U.S. application Ser. No. 09/705,377 entitled Spinal intervertebral Implant filed Nov. 3, 2000 in the name of Lawrence A. Shimp et al., and U.S. Pat. No. 6,277,149, all incorporated by reference herein.

During spinal surgery, the surgeon may approach the spine from a variety of different orientations. One orientation uses the posterior approach, another uses the anterior approach and others may approach laterally, posterior or anterior or antero-lateral, an angle somewhere between the anterior and lateral approaches.

U.S. Pat. No. 5,772,661 to Michelson discloses methods and instrumentation for surgical correction of the human thoracic and lumbar spine from the antero-lateral aspect of the spine. He states that interbody fusion has been performed from posterior, posterolateral and anterior which refers to the direction from which the bone grafts enter the intervertebral space. He further states that the straight anterior approach requires that the peritoneal cavity, which contains the intestines and other organs, be punctured twice, once through the front and once through the back on the way to the front of the spine, or secondly, by starting on the front of the abdomen off to one side and dissecting behind the peritoneal cavity on the way to the front of the spine. He states that there are at least two major problems specific to the anterior interbody fusion angle of implant insertion. First generally, at the L₄L₅ disc, the great iliac vessels that bifurcate from the inferior vena cava lie in close apposition to, and, covering that disc space make fusion from the front both difficult and dangerous. Second, anterior fusions have generally been done by filling the disc space with bone or by drilling across the disc space and then filling those holes with cylindrical implants. Present practice that is preferred is stated to place a ring of allograft femur into that disc space. However, to get good fill of the disc space places the sympathetic nerves along the sides of the disc at great risk. Alternatively, when dowels are used, because of the short path from the front of the vertebrae to the back and because of the height of the disc as compared to the width of the spine, only a portion of the cylindrical implant or implants actually engage the vertebrae, compromising the support.

Henry V.Crock, Practice of Spinal Surgery, pages 64-92, 1983, New York, N.Y., discloses the technique of anterior interbody fusion. FIG. 2.34 illustrates the use of retractors and swabs to protect the great vessels. FIG. 2.35 shows the L₄L ₅ levels. FIGS. 2.38, 2.42 and 2.43 illustrate some of the instruments used. FIGS. 2.46a and b illustrate some of the orientations of the grafts. FOR USE THEREWITH

U.S. Pat. No. 5,522,899 to Michelson discloses an implant insertion instrument in which the instrument has an implant engagement face that is concave that engages a convex face of the implant and includes an extension for engaging a depression in the implant face. The implant appears to be inserted from the anterior of the spine. The implant is shown as being inserted along the anterior-posterior axis of the spine.

U.S. Pat. No. 4,349,921 illustrates a cervical spinal implant and insertion instrument. The implant has a threaded bore and the instrument has a threaded stud which mates with the bore for inserting the implant along the posterior-anterior axis.

U.S. Pat. No. 4.878,915 to Brantigan illustrates the PLIF approach (posterior lumbar interbody fusion). He too uses a threaded implant and mating threaded insertion instrument for insertion of the implant along the posterior-anterior axis.

U.S. Pats. Nos. 5,192,327 and 5,425,772 also illustrate threaded implant bores for insertion by threaded insertion instruments. These too insert the implant straight into the disc space along a given axis and require the implant to have the desired shape and orientation for such insertion direction.

U.S. Pat. No. 5,645,598 shows an implant with an opening for receiving a screwdriver for inserting the implant. This too requires the implant to be inserted in a given orientation which must be accurate according to the implant configuration.

U.S. patent publication 2001/00221853 discloses a surgical instrument for applying implants. The instrument comprises a shank to which an implant holder is pivotally mounted and can be fixed in position. The implant holder is conical and threaded. The holder is threaded to an implant. The holder is fixed in an angular orientation by an adjustment of an axially displaceable rod. This instrument is awkward to use as once the implant is inserted the holder needs to be loosened so the holder and shank can be coaxial (aligned linearly) in order for the instrument to be unthreaded from the implant. An angle between the holder and shank would preclude rotation of the shank to unthread the holder from the implant due to limited space during surgery. This instrument is especially adapted to mate with a complementary threaded implant.

U.S. patent publications 2002/0022845 and 2002/0016592 illustrate still other instruments and implants for spinal surgery interbody fusion.

Paul M. Lin et al. in a text entitled Lumbar Interbody Fusion, chapter II directed to anterior lumbar spinal body fusion pages 127-131, Aspen publishers, Rockville, Md. 1989 discloses instrumentation and procedure for anterior spinal fusion.

An article in Spine, Vol.20, Number 24S, pp. 167 S-177 S entitled Interbody, Posterior, Combined Lumbar Fusions, discloses posterior, posterior lateral interbody fusions of interest.

Cloward, in an early work in 1952, entitled Lumbar Intervertebral Disc Surgery, Surgery, Vol. 32, No. 5, Nov. 1952, discloses lumbar intervertebral disc surgery employing a spreader.

Janssen et al. in an article entitled Biological Cages Eur Spine Jour (2000) 9 (Supp 1) pp. S102-109 discloses the femoral ring allograft and PLIF spacer used for anterior lumbar interbody fusion and posterior lumbar interbody fusion.

Other articles of interest in this field include Clinical Biomechanics of the Spine, by White et al. second ed. 1990, pp.547-563, Lippincott-Raven, New York, N.Y. showing various implant orientations, Campbell's Operative Orthopedics, Vol. Five, eighth ed, 1992, pp. 3505-3509 showing lateral approach and anterior approach spinal surgery, and a text Clinical Orthopaedics, Section II, General Orthopaedics, Anterior Lumbar Interbody Fusion by H.V. Crock, Lippincott Co.

Of interest are catalogs by DePuy Acromed VG2 Interbody Bone Grafts, 2000; Miltex Surgical Instruments, 1986 and Codman Surgical Products Catalog 1996, illustrating various surgical instruments.

However, the problem with the suggested techniques of the prior art is that the implants typically are oriented according to the orientation of their insertion. While many implants as disclosed are cylindrical dowels that mate in corresponding drilled holes in the spinal disc space, other spinal implants have generally flat surfaces, especially those for use in the lumbar spine or cervical spine. The latter implants may be derived from a transverse section of the diaphysis of a femur bone and typically are ring shaped with opposing sides that may be parallel or inclined to match the lordotic angles of the vertebral disc space. See for example the aforementioned copending application Ser. No. 60/372,972 filed Apr. 16, 2002 for examples of such implants. To properly fill the disc space, as recognized by the present inventors, requires such implants to have a shape and implanted orientation that is optimum for the disc space. Such implants may include a femoral ring allograft discussed above. These implants are fabricated from a slice section of bone taken from the diaphysis of a long bone such as the femur as discussed in the aforementioned application Ser. No. 60/372,972 and its corresponding utility application noted above.

These implants generally may overlie a relatively large intervertebral disc space area. These implants, however, if inserted in various anterolateral or lateral directions do not always have the optimum shape or orientation for the corresponding insertion direction. Such implants have insertion instrumentation receiving apertures, slots and bores and so on in relatively fixed orientation to the implant configuration. These apertures, slots and bores fix the orientation of the implant during insertion which orientation may not always be ideal. The surgeon thus has to improvise to deal with such implant misorientation.

Further, to manufacture a variety of different implants depending upon insertion orientation is wasteful, as it requires a large inventory of different relatively limited supply of bone implants of different configurations. This is because most present day implants have insertion instrument engaging apertures, slots or threaded bores for receiving the insertion instrument fixed thereto such as shown in the aforementioned U.S. publication 2001/00221853 and copending application Ser. No. 60/372,972 filed Apr. 16, 2002, for example. The orientation of the these instrument engaging apertures and so on thus determines the relative orientation and configuration of the implant due to the limitations of the surgeon's access region to the spinal region. That is, because regardless of the specific direction of insertion of the implant, the implant may have a configuration that is desired to fit in the mating intervertebral disc space, which is prefixed for the human spine, only varying generally in size, based for example, on the lordosis of the vertebrae and matching inclinations in the mating implant. Implants that have implanted orientations that ideally mate with the intervertebral space are believed most desirable.

One widely used implant design uses transverse slice sections of the diaphysis of a long bone such as the femur as discussed above. These slices tend to have cross sectional shapes that fit within the disc space in a predetermined orientation, especially if the lordotic angles of the implant surfaces are also formed into the implant, whether lumbar or cervical or otherwise, usually with some bone processing to finish the implant final configuration.

Also, during insertion, the vertebrae are bumpy and during insertion the bumps tend to force the implant into undesired orientations. During surgery, the surgeon needs to improvise on this problem and use what ever instruments that may be available for reorienting the implant. Such instruments may be for implant insertion, tamps, curettes, trials, rasps and so on which are designed for specific processing steps and not for orienting the implants. These instruments may, as a result, damage the bone implant, e.g., fracture or splinter it. As recognized by the present inventors, the instruments are not complementary to the implants and also are difficult to use for this purpose because that is not their design function.

Another problem is that during insertion of the implant, the vertebrae are distracted by a distractor instrument that typically remains in place during implant insertion. The implant insertion instrument then is used to insert the implant in the presence of the distractor. In anterior approach or antereolateral approach, because the great vessels and various organs are present, there is little room for the surgeon to manipulate the implant, especially with implant insert instruments that comprise a set of jaws and due to the presence of the other instruments such as a vertebral spreader or distractor. Also, there is little room for the surgeon to observe accurately the position of the implant. After removal of the insertion instrument, and possibly the distractor instrument, the surgeon then has an improved view of the inserted implant and then may determine that the implant is not in the ideal orientation. This is due to the fact that the implant has superior and inferior surfaces that in many instances are inclined to match the lordosis of the vertebral space and thus has only one desired orientation relative to the anterior-posterior axis.

It is at this time the surgeon looks around his instrument kit for something to use to reorient the implant, such instruments not being appropriate for such reorientation. Such tools may damage the implant at the bore usually present for an insertion instrument and the implant at the bore is relatively weak. For example, many bone implants in the prior art have bores or recesses used in conjunction with mating insertion implant engagement members or jaws. These bores or recesses may be used by the surgeon for insertion of a non-compatible instrument for reorienting the implant. Such instruments typically subject the implant to possible stress damage to the bone at such weakened locations created by such bores and recesses. The surgeon may insert the instrument into such bore or recess and tap on the instrument with a hammer damaging the implant.

Because of the incompatibility of such instruments with such orienting maneuvers, such tapping introduces additional compressive and/or bending stress forces on the bone at the point of contact, which in practice may be just a contact point, and not a torque spread over an area as desired. The bone may chip or fracture at such stress points.

A need is seen by the present inventors for a solution to this problem. A need is seen in particular for instrumentation and implants for the reorientation of an installed spinal implant in the intervertebral space that will enable a physician to correct for the above described misorientation of the implant without damage and which instrument does not take up a significant amount of space in the available surgically created body cavity during use.

An instrument according to the present invention is for manipulating and reorienting a bone spinal implant inserted in the intervertebral disc space of a spine. The disc space defines a plane. The implant is for being inserted into the disc space in a range between the anterior to lateral approaches in the plane and has a smooth surfaced cylindrical bore therein of a given diametrical dimension and longitudinal extent. The instrument comprises an elongated shaft defining a first longitudinal axis, the shaft having a proximal end and a distal end. A handle is at the distal end for manipulating the shaft. An implant manipulating substantially smooth surface implant engaging cylindrical member extends from the proximal end, the engaging member defines a second longitudinal axis which preferably is inclined relative to the first longitudinal axis, but may be coaxial therewith, the engaging member being dimensioned for matingly engaging the implant bore and for manipulating the implant in the plane of the disc space in response to a force on the handle while imparting substantially only a torque on the implant with negligible stress concentration.

As a result, the instrument can be rotated or otherwise manipulated to move the implant in the plane of the disc space to the implant desired orientation relative to the anterior-posterior axis of the spine. By making the implant bore cylindrical and smooth surfaced and the instrument bore engaging member cylindrical and smooth surfaced and dimensioned to closely engage the bore, the surgeon can tap on the handle of the instrument with a hammer, for example, to torque and rotate or otherwise reorient the implant to its preferred orientation. This permits the implant to be manipulated without exposing the relatively delicate bone structure of the implant to undesirable stress concentrating fracturing or distortion forces. Such fracturing or distortion might otherwise occur if the instrument is not specially designed to mate in the implant bore. This may require the removal and replacement of a damaged implant, which is costly and undesirable and subjects the patient to additional trauma.

The implant that is a slice of a femur bone is typically annular with a central bore formed by the medullary canal which may be further finished. The implant vertebral bearing surfaces are roughened, for example with teeth and the like and provided with lordotic angles. Thus the implant is shaped to have a given orientation with the spinal anterior-posterior axis. If the implant is inserted from an anterolateral approach or other angle to the anterior-posterior axis, and is not aligned appropriately to the spine, it may need to be reoriented so that its anteriorposterior posterior axis is aligned with the spinal anterior-posterior axis. The surgeon can so manipulate the implant by inserting the bore engaging member into the implant bore and then manipulate implant with the instrument without stress inducing fracturing of the implant.

In one aspect, the implant engaging member has an axial extent and a diameter relative to the diameter of the implant bore such that the implant displaces in unison with the implant engaging member in response to a displacement force on the handle.

Preferably, the implant engaging member and the bore have substantially the same complementary cross sectional shape for the length of the bore and for the length of the implant engaging member engaged with the bore.

In a further aspect, the shaft has a first portion extending a first longitudinal axis and a second portion extending along a second longitudinal axis, the implant engaging portion forming an extension of the second portion.

In a still further aspect, the shaft has a first transverse dimension and the implant engaging portion has a second transverse dimension smaller than the first transverse dimension.

In a further aspect, the implant engaging portion has an end tip surface distal the shaft, the end tip surface being rounded, spherical or flat.

In a further aspect, a spinal bone implant according to the present invention comprises a body made of bone lying in a plane and having superior and inferior surfaces for bearing against respective adjacent vertebrae defining a disc space therebetween, the body having spaced respective anterior and posterior end surfaces defining an anterior-posterior axis, the body having a bore in communication with an end surface at the anterior end and extending in the region between the inferior and posterior surfaces, the bore being at least one of inclined at an angle to the anterior-posterior axis in the implant plane or offset relative to the anterior-posterior axis in the implant plane.

In accordance with a further aspect, the invention comprises the combination of the instrument set forth above with any of the implants set forth above.

### IN THE DRAWING:

FIG. 1 is a side elevation view of a spinal fusion implant adjustment instrument according to an embodiment of the present invention;

FIG. 2 is a sectional plan view of the human spinal bone implant of FIG. 5 taken at lines 2-2;

FIGS. 2a, 2b and 2c are sectional plan views similar to that of FIG. 2 according to further embodiments of the implant of the present invention;

FIG. 3 is a plan view of a cortical bone implant taken from a transverse section of the diaphysis of a femur bone for use with the instrument of FIG. 1;

FIG. 3a is an isometric view of the implant of FIG. 3;

FIG. 4 is an anterior end elevation view of the implant of FIG. 3 taken at lines 4-4;

FIG. 5 is a side elevation view of the implant of FIG. 3 taken at lines 5-5;

FIG. 6 is a plan sectional view of a human lumbar spine and fragmented view of the instrument of FIG. 1 showing the implant of FIG. 3 after insertion in an antereolateral orientation;

FIG. 7 is a plan sectional view of a human lumbar spine showing the implant of FIG. 6 after it is reoriented to the proper anterior-posterior axis using the instrument of FIG. 1;

FIG. 8 is a plan sectional view of the spine showing the engagement of the instrument of FIG. 6 with the implant prior to reorienting the implant;

FIG. is a sectional view of a portion of an implant at the instrument receiving bore portion showing a further embodiment of the implant;

FIG. 10 is a sectional view of a portion of an implant at the instrument receiving bore portion showing a further embodiment of the implant wherein the broken lines show further variations in the instrument receiving bore;

FIGS. 11, 12 and 13 are sectional views of a portion of an implant and the mating orienting instrument tip showing further embodiments of the instrument implant engaging tip and its relationship to the implant bore for illustration of the principles of the present invention; and

FIGS. 14 and 14a are more detailed side elevation views of the adjustment instrument in more detail according to further embodiments.

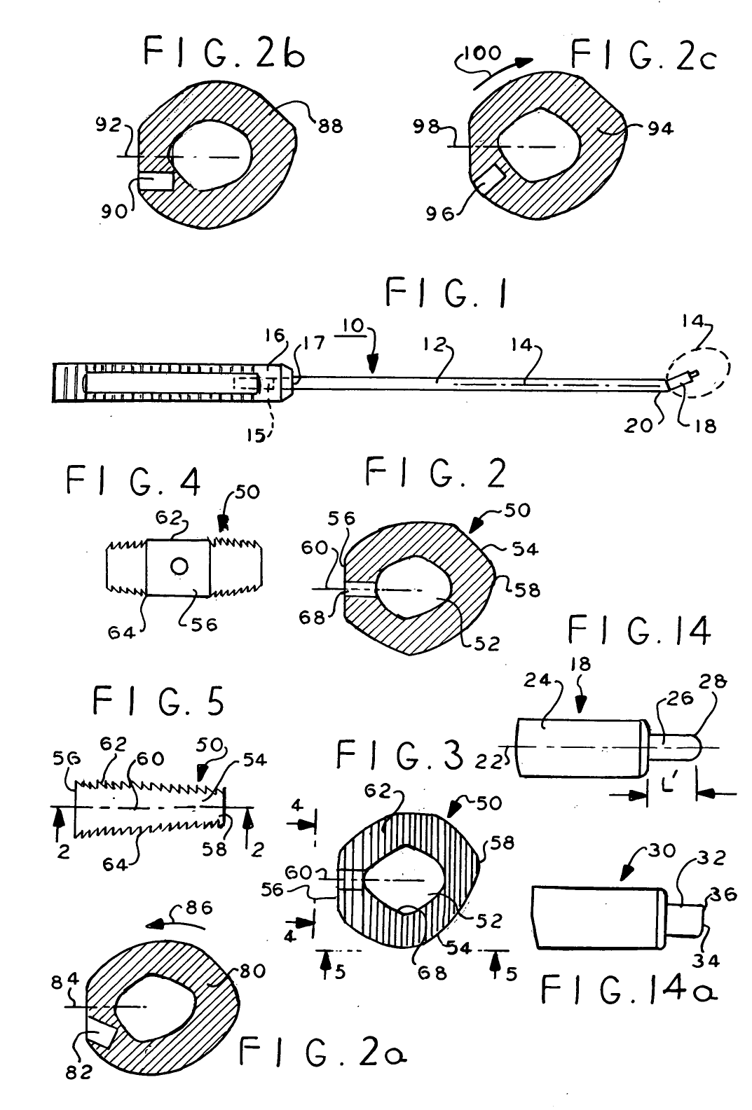

In FIG. 1, instrument 10 comprises an elongated shaft 12 defining an elongated longitudinal axis 14, a handle 16 attached to the shaft 12 at the shaft distal end 17 and an implant manipulating and engaging member 18. The handle 16 may have a bore 15 in which the shaft 12 is inserted and attached to the handle. The shaft 12 may be press fit into the handle bore 15 in interference fit, may be threaded to the handle at bore 15 or may be bonded to the handle such as by welding. The handle 16 is elongated and extends along the axis 14.

The handle 16 has a transverse dimension greater than that of the shaft 12 to permit ease of gripping by a surgeon during use. The handle and shaft may be formed of stainless steel, for example, and preferably is circular cylindrical or, in the alternative may have other cross section shapes such as square or rectangular, for example. The handle 16 may also have flattened surfaces (not shown) for receiving hammer blows used to manipulate the instrument 10 for manipulating an implant inserted into the intervertebral disc space as will be explained below. The shaft proximal end 20 is preferably bent to form implant engaging member 18 from the shaft 12. Member at an angle _{α}, FIG. 6, and which angle may have a value in the range of about 30-70°.

Member lines longitudinal axis 22 which is at angle a to longitudinal axis 14. The shaft 12 on axis 14 forms a first shaft portion and the portion on axis 22 forms a second shaft portion 24. The shaft second portion 24 has an extension which forms an implant engaging portion 26. Portion 24 extends along axis 22 from the shaft 12 first portion. The shaft second portion 24 has length L, FIG. 6, which may be any desired value and preferably about 10-50 mm (0.5-2 inches).

The implant engaging portion 26 is an extension of and coaxial with portion 24. The implant engaging portion 26, FIG. 14, has a length L′ which is determined as explained below in more detail. The portion 26 along its length L′ is preferably circular cylindrical in transverse section to match the shape of the mating implant bore as will be explained. The portion 26 has an end tip surface 28 which is rounded and preferably spherical. However, the end tip surface may be planar in the alternative as shown for implant engaging member 30, FIG. 14a, which has an implant engaging portion 32. The portion 32 has a flat tip end surface 34 with slightly rounded edge 36 formed by a radius. In a still further alternative, the implant engaging portion may extend for its entire length at angle α from the shaft 12 portion. The length and other parameters of the implant engaging member configuration may be determined empirically according to a given implementation.

For example, FIG. 11 illustrates an implant engaging portion 38 which is relatively short with a spherical tip surface 40. FIG. 12 illustrates an implant engaging portion 42 which is relatively longer than portion h a flat tip surface 40 normal to its longitudinal axis 43. In FIG. 13, the implant engaging portion 44 is relatively longer than portion smaller in diameter than portion 42. The implant engaging portions of FIGS. 11, 12 and 13 are all shown in engagement with a bore 46 of an implant of the same bore diameter. The advantages and disadvantages of the relative differences of the portions 38, 42 and 44 to the bore diameter will be explained below.

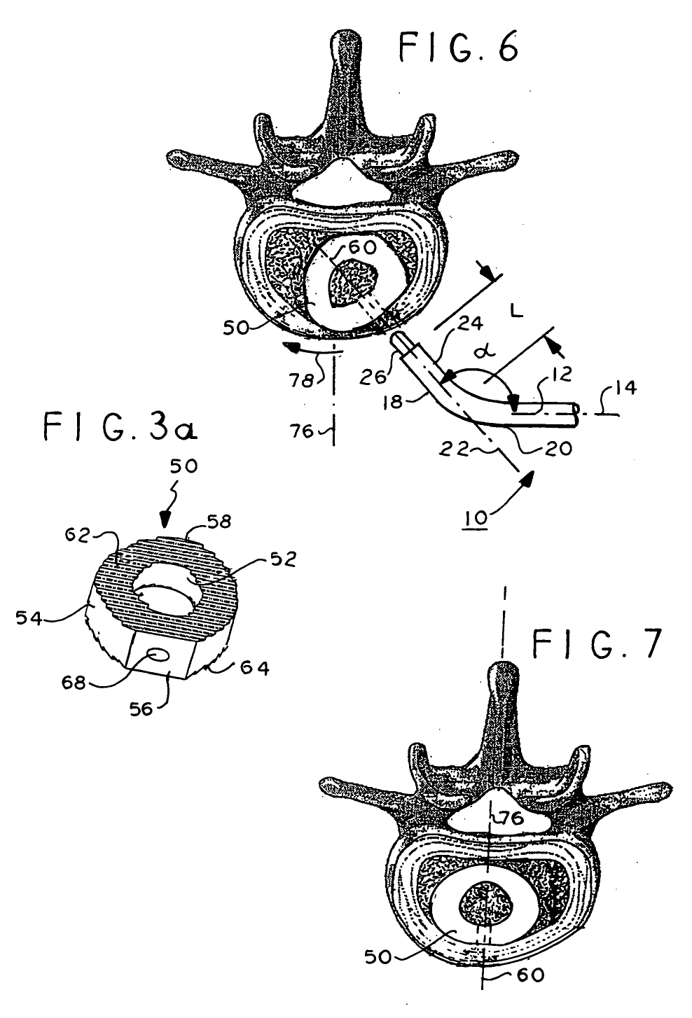

A representative implant 50, FIGS. 3 and 3a is made of cortical bone and formed from a transverse section slice taken from the diaphysis of a long bone such as the femur or tibia. The implant 50 has a central chamber 52 which may originally be formed from the medullary canal of the long bone. The interior wall surface of the chamber 52 is preferably removed to leave cortical bone with the marrow and other contaminants removed. The implant 50 has an outer peripheral surface 54 which is generally curved as shown as formed by the natural long bone from which the implant is taken. The implant 50 has an anterior end 56 and a posterior end 58, the latter being part of the curved peripheral surface 54. The surface outer peripheral surface at end 56 is planar and normal to the implant anterior-posterior axis 60. The implant 50 has a superior surface 62 and an inferior surface 64, FIGS. 4 and 5. Surfaces 62 and 64 are both serrated with parallel saw teeth which run normal to the axis 60. The anterior end 56 has a height greater than the posterior end 54.

Bore(s) or recesses (not shown) may be located in the implant peripheral surface 54. The bores or recesses are used to receive the gripping jaws of an implant insertion instrument (not shown) as known in this art. See for example, several of the copending applications and patent mentioned in the introductory portion incorporated by reference in their entirety herein.

The implant 50 has a bore receiving the implant engaging portion 26, FIG. 14. It is preferred that the portion 26 have length L′, FIG. 14, that is sufficient to permit the instrument 10 to rotate manipulate the implant 50 in the plane of the implant as defined by axis 60, FIG. 5, normal to the plane of the drawing sheet. To this end, it is preferred that the diameter of the bore such as to closely receive the implant engaging portion 26 as best seen in FIG. 12 as represented by implant engaging portion 42. This spacing of the engaging portion 26 to the bore such that any rotation of the instrument 10 imparts only a torque on the implant with minimum stress concentration on the implant at the bore. By closely matching the portion 26 to the bore 68, the instrument and implant will rotate in unison with no play therebetween. Any play therebetween will result in tilting the engaging portion relative to the bore and result in stress concentration at a localized point or region rather than spreading the displacement force over a relatively wider area of the bore. Such spreading action of the force minimizes stress concentration and thus minimizes potential damage to the bone implant.

Apertures and bores and recesses normally used for insertion instruments are thus not satisfactory. This is because of the lack of match of a randomly selected instrument to implant recesses or bores used for insertion of the implant.

Portion 42 has a length that extends for a sufficient depth into the bore 46 and is closely received, so that the implant places one-for-one with a displacement of the instrument implant engaging portion 42. This insures minimum stress concentration on the bone implant at a relatively weak area at the bore. This also provides optimum control by the surgeon of the direction and amount of displacement of the implant.

If for example, the implant engaging portion 38, FIG. 11, were related to the implant bore such as bore 46, FIG. 11, a displacement of the portion 38 in direction 70 would only rotate the portion 30 intially due to play between the portion 38 and the bore 46 created by the clearance 72 between the portion 38 and the bore 46 surface. Also, the portion 38 would tend to rotate within the bore 46 due to this clearance as well. This will produce undesired stress concentration on the bone of the implant at point 73 when the instrument is rotated in direction 70. This stress concentration could fracture the bone at the edge of the bore 46 with anterior surface 56. Also, displacement of the instrument does not result in a corresponding displacement of the implant. Further, the relative impact rotation of the portion 38 within the bore 46, especially in response to an impact force on the instrument, might also accelerate at time of contact with the bore surface at point 73, increasing the stress concentration which may also fracture or otherwise damage the bone of the implant.

While the portion displace the implant when the instrument handle is displaced carefully, such displacement of the implant is not optimum. The relative dimensions between the portion the bore, i.e., the clearance and foreshortened length of the portion 38, are not desirable for optimum control of manipulation of the implant and to minimize implant damage. A relative length and close fit between the portion 42 and bore 46, FIG. 12, is recommended as shown in FIG. 12. In any case, regardless of the care taken, stress concentration will occur at point 73.

The desired close fit, e.g., in the order of a mm or fraction thereof, at clearance 72 as shown in FIG. 12, minimizes wobble of the portion 42 when it is displaced, thus tending to move the implant in unison with motions of the portion 42 in response to instrument displacement. This also removes the possible acceleration and localized concentrated impact forces of the portion 42 with the bore surface 46. The torque force is spread over the length of the portion 42 and surface of the bore 46.

In FIG. 13, while the portion 44 is longer than portion 38, FIG. 11, the portion 44 also exhibits undesirable clearance 74. This clearance, which may be acceptable, depending upon its value relative to the engagement length of the portion 44 to the bore 46 surface, also is not ideal as it may result in relative motion between the portion 44 and the bore 46 surface and damage the bone at point 75.

The amount of clearance and the relative length L′ of the implant engaging portion 42, FIG. 12 and portion 26, FIG. 14, may be determined empirically to obtain he acceptable differences therebetween for each implementation. Such determination may be made by one of ordinary skill in this art.

It is important that the implant bore the implant engaging portion 26 be smooth surfaced to optimize coupling between of the portion 26 to the bore 68. Threads or roughness in the surfaces may become damaged due to stress concentration at the thread crests, especially if the portion 26, FIG. 14, were either threaded or smooth if the bore 68, FIG. 2, were threaded or otherwise rough. The portion 26 when impacted against the threads or roughened surface of the implant bore at points such as 73, FIG. 11, or 75, FIG. 13, could easily compress and damage the bone at these locations, which is not desirable and could destroy the implant.

In operation, in FIG. 6, the instrument 10 is placed adjacent to the inserted implant. As seen, the implant 50 (its surface, roughness not shown for clarity of illustration), with its lordotic surfaces 62, 64 (FIG. 5) is inserted with its anterior-posterior axis 60 misoriented relative to the spinal anterior-posterior axis 76. It is required that the implant 50 be rotated in direction align its anterior-posterior axis 60 with the spinal anterior-posterior axis 76. The misorientation of the implant is exaggerated for purposes of illustration. The implant may be just slightly misaligned from the anterior-posterior axis 76 according to each relevant surgical procedure.

In FIG. 7, the implant 50 (its surface roughness not shown for clarity of illustration) is shown properly oriented with its anterior-posterior axis 60 aligned with the spinal anterior-posterior axis 76.

In FIG. 2a, bone implant 80 is shown with an instrument receiving bore 82 inclined and offset from the implant's anterior-posterior axis 84. The other parameters of the implant 80 may be the same as that of implant 50, FIG. 3a. Such inclination and offset are introduced to facilitate reorientation of the implant for certain implementations in order to facilitate the rotation of the implant by the instrument 10 for a given implant insertion orientation. The bore 82 orientation is to facilitate rotation of the implant 80 in direction 86.

In FIG. 2b, bone implant shown with an instrument receiving bore 90 parallel to but offset from the implant's anterior-posterior axis 92. The other parameters of the implant be the same as that of implant 50, FIG. 3a. Such offset is introduced to facilitate reorientation of the implant for certain implementations in order to facilitate the rotation of the implant by the instrument 10 for a given implant insertion orientation.

In FIG. 2c, bone implant 94 is shown with an instrument receiving bore 96 inclined and offset from the implant's anterior-posterior axis 98. The other parameters of the implant 94 may be the same as that of implant 50, FIG. 3a. Such inclination and offset are introduced to facilitate reorientation of the implant for certain implementations in order to facilitate the rotation of the implant by the instrument 10 for a given implant insertion orientation. The bore 96 orientation is to facilitate rotation of the implant 94 in direction 100.

FIG. 9 illustrates an implant 102 having a blind instrument receiving bore 104 of one depth more than 50% of the thickness of the wall 106 to the central chamber wall 108. FIG. 10 illustrates an implant 110 having a blind instrument receiving bore 112 of a depth less than 50% of the thickness of the wall 114 to the central chamber wall 116. The dashed line 118 range of possible depths for the bore 112 in different embodiments according to a given implementation.

It will occur to one of ordinary skill that modifications may be made to the disclosed embodiments without departing from the scope of the invention as defined in the appended claims. The disclosed embodiments are given by way of illustration and not limitation. For example, the implant engaging member while shown one piece with the instrument shaft 12 may be two pieces therewith and assembled to the shaft with threads or other mechanical coupling arrangements, whether fixed, movable or releasable.

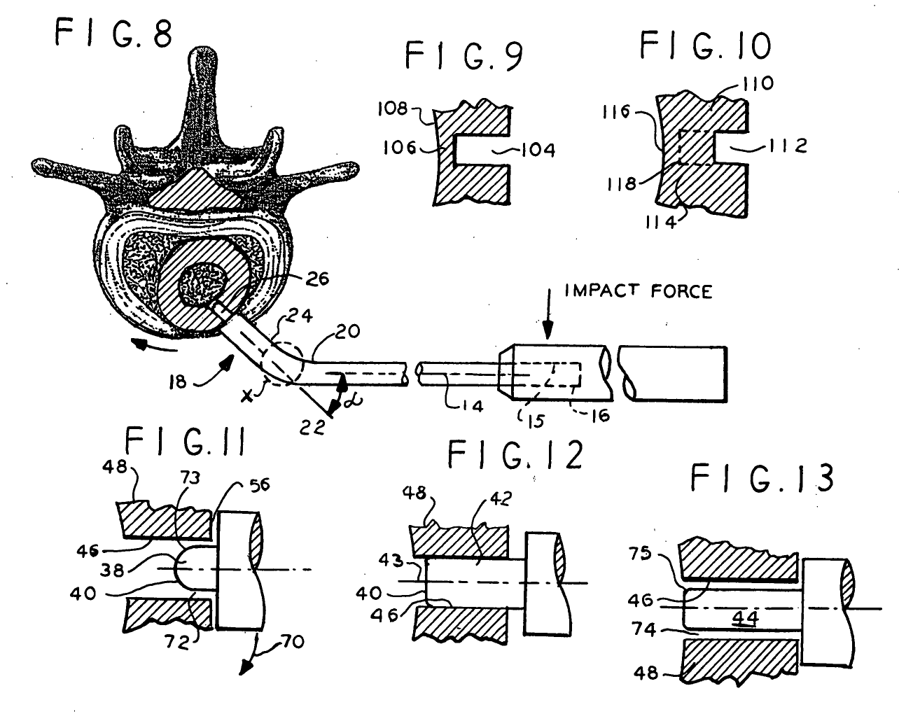

By way of further example, the implant engaging member may be affixed to a ball joint that is selectively loosened and tightened to the shaft. This permits the member to be oriented at any desired angle to the shaft. Such a ball joint may be in region X, FIG. 8, and may be constructed as disclosed in the aforementioned U.S. patent publication 2001/00221853 incorporated by reference herein. This publication discloses an implant insertion device that is especially adapted to a particularly threaded bore in the implant. While that insertion device might be used for manipulation of the corresponding implant, it has no general use with other implants unless they are all threaded the same way. In the present invention, various implants for engagement with various different insertion instrument, all could be manipulated by a common single instrument regardless the insertion instrumentation configuration which may differ for different implants.

What is claimed is:

1. A spinal implant insertion adjustment instrument for manipulating and reorienting a spinal implant made of bone inserted in the intervertebral disc space of a spine, the disc space defining a plane, the implant for being inserted into the disc space in a range between the anterior to lateral approaches in the plane and having a bore therein of a given smooth surface diametrical dimension and longitudinal extent, the instrument comprising:
an elongated shaft defining a first longitudinal axis, the shaft having a proximal end and a distal end;
a handle at the distal end for manipulating the shaft; and
an implant manipulating substantially smooth surface implant engaging member extending from the proximal end, the engaging member defining a second longitudinal axis at an angle in the range of at least 0° to a maximum inclination relative to the first longitudinal axis, the engaging member being dimensioned for matingly engaging the implant bore and for manipulating the implant in the plane of the disc space in response to a force on the handle.

2. The instrument of claim 1 wherein the implant bore and the implant engaging member are each circular cylindrical.

3. The instrument of claim 2 wherein the implant engaging member diametrical dimension is dimensioned to be closely received in the implant bore to minimize stress concentration of the engaging member against the implant bore surface.

4. The instrument of claim 2 wherein the implant engaging member has an axial extent and a relative diameter to the diameter of the implant bore such that the implant displaces in unison with the implant engaging member in response to a displacement force on the handle.

5. The instrument of claim 1 wherein the implant engaging member and the bore have substantially the same cross sectional shape for the length of engagement therebetween.

6. The instrument of claim 4 wherein the implant engaging member is closely received in the bore to minimize impact concentrated acceleration forces on the implant by the instrument.

7. The instrument of claim 1 wherein the shaft has a first portion extending along said first longitudinal axis and the a second portion extending along said second longitudinal axis, the implant engaging portion forming an extension of said second portion.

8. The instrument of claim 7 wherein the shaft has a first transverse dimension and the implant engaging portion has a second transverse dimension smaller than the first transverse dimension.

9. The instrument of claim 1 wherein the implant engaging portion has an end tip surface distal said shaft, the end tip surface being rounded.

10. The instrument of claim 9 wherein the end tip surface is spherical.

11. The instrument of claim 1 wherein the implant engaging portion has an end tip surface distal the shaft and an outer surface, the end tip surface being planar and transverse to the second axis and coupled to the engaging portion outer surface by a curved portion.

12. The instrument of claim 1 wherein the second axis is oriented in the range of about 30-70° to the first axis.

13. An instrument for manipulating and reorienting a spinal implant inserted in the intervertebral disc space of a spine, the disc space defining a plane, the implant for being inserted into the disc space in a range between the anterior to lateral approaches in the plane and having a circular cylindrical smooth surface bore therein of a given diametrical dimension and longitudinal extent, the instrument comprising:
an elongated shaft defining a first longitudinal axis, the shaft having a proximal end and a distal end;
a handle at the distal end for manipulating the shaft;
a shaft extension extending from the proximal end defining a second longitudinal axis inclined relative to the first longitudinal axis; and
an implant manipulating substantially smooth surface implant engaging member extending from the shaft extension a second longitudinal extent, the smooth surfaced implant engaging member being dimensioned for matingly engaging the implant bore for manipulating the implant in the plane of the disc space in response to an impact force on the handle to minimize damaging stress concentration on the implant in the bore.

14. An instrument for manipulating and reorienting a spinal implant inserted in the intervertebral disc space of a spine, the disc space defining a plane, the implant for being inserted into the disc space in a range between the anterior to lateral approaches in the plane and having a circular cylindrical smooth surfaced bore therein of a given diametrical dimension and longitudinal extent, the instrument comprising:
an elongated shaft defining a first longitudinal axis, the shaft having a proximal end and a distal end;
a handle at the distal end for manipulating the shaft;
a shaft extension extending from the proximal end defining a second longitudinal axis inclined relative to the first longitudinal axis; and
an implant manipulating circular cylindrical substantially smooth surface implant engaging member extending from the shaft extension a second longitudinal extent, the circular cylindrical smooth surfaced implant engaging member being dimensioned along said second longitudinal extent for matingly engaging the implant bore over at least a portion of the first longitudinal extent, the instrument for manipulating the implant in the plane of the disc space with minimum stress concentration on the implant in the bore in response to an implant displacing impact force on the handle.

15. An instrument and spinal bone implant for insertion into the intervertebral disc space of a spine, the instrument for manipulating and reorienting the implant after insertion into the disc space, the disc space defining a plane, the instrument and implant comprising:
a bone implant having an anterior end surface and a posterior end surface defining an anterior-posterior axis, a superior surface for bearing against a first vertebra and an inferior surface for bearing against a second vertebra defining the disc space, the implant having a bore therein of a given orientation relative to the anterior-posterior axis, the bore having a smooth surface and a diametrical dimension and a longitudinal extent between and in a direction generally along and spaced from said superior and inferior surfaces in said given orientation; and
said instrument for manipulating the implant at said bore comprising:
an elongated shaft defining a first longitudinal axis, the shaft having a proximal end and a distal end;
a handle at the distal end for manipulating the shaft; and
an implant manipulating substantially smooth surface implant engaging member extending from the proximal end, the engaging member defining a second longitudinal axis inclined relative to the first longitudinal axis, the engaging member being dimensioned for matingly engaging the implant bore to minimize stress concentration on the implant in the bore and damage to the implant in the bore and for manipulating the implant in the plane of the disc space in response to a manipulation force on the handle.

16. The instrument and spinal implant of claim 15 wherein the bore is offset from said axis.

17. The instrument and annular spinal implant of claim 15 wherein the bore is inclined at an angle to the axis.

18. The instrument and annular spinal implant of claim 15 wherein the bore is blind.

19. The instrument and spinal implant of claim 15 wherein the implant has a central opening transverse to the axis and in communication with the superior and inferior surfaces, the bore extending through the implant into communication with said opening.

20. The instrument and spinal implant of claim 20 wherein the bore and instrument have mating surfaces that are closely spaced.

21. A spinal bone implant comprising:
a body made of bone and having superior and inferior surfaces for bearing against respective adjacent vertebrae defining a disc space therebetween, the body defining a plane and having spaced respective anterior and posterior end surfaces defining an anterior-posterior axis in the plane, the body having a bore in communication with an outer peripheral surface at the anterior end surface and extending in the region between the inferior and posterior surfaces, the bore being at least one of inclined at an angle to the anterior-posterior axis or offset relative to the anterior-posterior axis.

22. The implant of claim 21 wherein the bore is blind.

23. The implant of claim 21 wherein the implant has a central opening in communication with said inferior and superior surfaces, the bore being blind and extending in a direction parallel to the inferior and superior surfaces.

24. The implant of claim 21 wherein the inferior and superior surfaces are inclined relative to each other to mate with the lordosis of the spine.

25. The implant of claim 21 wherein the implant has a central opening in communication with said inferior and superior surfaces, the bore being in communication with the anterior end surface and the central opening.

26. The implant of claim 21 wherein the implant has an outer peripheral wall surface, the outer peripheral wall surface having a curved portion and a flat portion, the flat portion being located on said axis at said anterior end of the implant.

27. The implant of claim 26 wherein the bore is in communication with said flat surface portion.

28. The implant of claim 26 wherein the bore is inclined relative to the flat surface portion.

29. The implant of claim 26 wherein the bore is offset from said axis.

30. The implant of claim 21 wherein at least one of the inferior and superior surfaces are roughened to minimize backing out of the implant from between the vertebrae and at least one of the inferior and superior surfaces is inclined relative to the axis.

31. The implant of claim 21 wherein the body is made of a section of the diaphysis of a long bone.

32. The implant of claim 21 wherein the body is cortical bone.

33. In combination:
a spinal bone implant manipulating instrument comprising:
an elongated shaft defining a first longitudinal axis, the shaft having a proximal end and a distal end;
a handle at the distal end for manipulating the shaft; and
a circular cylindrical implant bore engaging member having a substantially smooth surface and extending from the proximal end, the engaging member defining a second longitudinal axis inclined relative to the first longitudinal axis, the engaging member being dimensioned for matingly engaging the implant bore set forth below and for manipulating the spinal bone implant set forth below in the plane of the disc space in response to a force on the handle while minimizing stress concentration on the implant;
the implant comprising a body made of bone and having superior and inferior surfaces for bearing against respective adjacent vertebrae defining a disc space therebetween and in movable engagement with the vertebrae in the disc space plane, the body having spaced respective anterior and posterior end surfaces defining an anterior-posterior axis, the body having a smooth surface cylindrical bore in communication with the anterior end surface for receiving the bore engaging member in close complementary engagement to minimize stress concentration on the implant bore, the bore extending between the inferior and posterior surfaces.

34. The combination of claim 33 wherein the bore in the body is at least one of inclined at an angle to the anterior-posterior axis and offset relative to the anterior-posterior axis.
